(19) United States
(12) Patent Application Publication
(10) Pub. No.: US 2024/0173165 A1
(43) Pub. Date: May 30, 2024

(54) Algorithm Using Common Patient Parameters to Determine Correct Pad Size

(71) Applicant: C. R. Bard, Inc., Franklin Lakes, NJ (US)

(72) Inventors: Jesse Smith, Broomfield, CO (US); Niharika Pathare, Thane (IN); Gabriel A. Johnston, Broomfield, CO (US); Mengjia Yi, Westminster, CO (US)

(21) Appl. No.: 18/282,748

(22) PCT Filed: Mar. 16, 2022
(86) PCT No.: PCT/US22/20633

§ 371 Date: Sep. 18, 2023

Related U.S. Application Data
(60) Provisional application No. 63/162,955, filed on Mar. 18, 2021.

(51) Int. Cl. A61F 7/08; A61F 7/00; G16H 10/60; G16H 20/30; G16H 50/70

(52) U.S. Cl. CPC A61F 7/08; A61F 7/0085; G16H 10/60; G16H 20/30; G16H 50/70; A61F 2007/0018; A61F 2007/0041; A61F 2007/0054

(57) ABSTRACT

A system is disclosed for recommending a thermal pad set for use in providing a targeted temperature management (TTM) therapy to a patient, where the system includes a computer implemented method. The computer implemented method includes receiving a request for a thermal pad set recommendation from a clinician device, receiving a patient's identification from the clinician device, retrieving one or more patient parameter values from an electronic medical record for the patient, determining a pad set recommendation according to the patient parameter values in combination with a pad set correlation table, and displaying the pad set recommendation on the clinician device. Also disclosed herein is a system including a non-transitory computer-readable medium with instructions encoded thereon and one or more processors configured to, when executing the instructions, perform operations in accordance with processes of the computer implemented method.

### PRIORITY

This application claims the benefit of priority to U.S. Provisional Application No. 63/162,955, filed Mar. 18, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled with a patient to affect surface-to-surface thermal energy exchange with the patient. In general, TTM systems comprise a TTM fluid control module coupled with at least one contact pad via a fluid deliver line. One such TTM system is disclosed in U.S. Pat. No. 6,645,232, titled “Patient Temperature Control System with Fluid Pressure Maintenance” filed Oct. 11, 2001 and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain thermally intimate pad-to-patient contact is of importance to fully realizing medical efficacies with TTM systems.

In some instances, multiple thermal pad sizes are available to accommodate a wide range of patient sizes. To maximize thermal energy exchange with the patient, it may be advantageous to match the thermal pad size to the patient size. As a patient size may be defined by different characteristics such as weight and height, choosing the thermal pad size may require combining the different patient size characteristics is a specific manner to arrive at the optimal pad size. Furthermore, the optimal pad size may not be readily available, in which case the optimal second choice pad size may need to be used. Disclosed here are systems and method for choosing the optimal thermal pad size for a given patient from an available inventory of the thermal pads.

### SUMMARY OF THE INVENTION

Briefly summarized, disclosed herein is a system and computerized method for automatically determining a recommended thermal pad set for use in providing a targeted temperature management (TTM) therapy to a patient. In one embodiment, the computerized method includes receiving a request for a thermal pad set recommendation for an identified patient from a clinician device, receiving a patient's identification from the clinician device, accessing an electronic medical record (EMR) for the patient, retrieving one or more patient parameter values from the EMR, determining a pad set recommendation according to the patient parameter values in combination with a pad set correlation table, and displaying the pad set recommendation on the clinician device.

In some embodiments, the pad set includes at least one torso pad and the pad set may also include at least one thigh pad. The patient parameters may include at least two of the patient's gender, weight, height, or body fat percentage. In some embodiments, the patient parameters include at least three of the patient's gender, weight, height, or body fat percentage. Additionally, in some embodiments, the patient parameters may include a plurality of predetermined body shapes, where each body shape may correspond to body measurement ranges or body fat percentage ranges that are used by the computerized in providing a thermal pad set recommendation. Additionally, or alternatively, each body shape may correspond to expected locations for body fat deposits, which may influence the thermal pad set recommendation provided by the computerized method. For example, a patient with a “pear-shaped”body may be expected to have a greater accumulation of body fat around the patient's waist and hip regions than a patient with an “inverted-triangle-shaped” body.

The computerized method may further include receiving one or more other patient parameters from the clinician device and the other patient parameters may include at least one of the patient's pant waist size, pant inseam size, or shoe size. In some embodiments, the other patient parameters include at least two of the patient's pant waist size, pant inseam size, or shoe size.

In some embodiments, determining the pad set recommendation includes determining an initial pad set recommendation in accordance with a first set of patient parameters and determining a refined pad set recommendation in accordance with a second set of patient parameters in combination with the first set of patient parameters. In such embodiments, rendering the pad set recommendation on the clinician device includes rendering refined pad set recommendation. In some embodiments, the refined pad set recommendation is different from the initial pad set recommendation.

The first set of patient parameters may include one or more of the patient parameters, and the second set of patient parameters may include one or more of the other patient parameters. The first set may include the patient's weight and/or the patient's height and the second set of patient parameters may include the patient's pant waist size and/or the patient's pant inseam size.

The computerized method may further include accessing a facility inventory system and determining an availability of the recommended pad set in inventory. In some embodiments, if the pad set is not available in inventory, the computer implemented method further includes determining an alternative pad set and displaying the alternative pad set on the clinician device.

The computerized method may also include determining the recommended thermal pad set according to the patient parameter values using a trained machine learning model, where the trained machine learning model receives as input the one or more patient parameter values and provides one or more resultant scores, and where a highest resultant score is provided as the recommended thermal pad set.

Also disclosed herein is a system including one or more processors and a non-transitory computer-readable medium communicatively coupled to the one or more processors and having instructions stored thereon that, when executed by the one or more processors, cause performance of operations in accordance with processes of the computerized method summarized above.

Also disclosed herein is a non-transitory computer-readable storage medium (CRM) including executable instructions that when executed by one or more processors causes the one or more processors to perform operations in accordance with processes of the computerized method summarized above.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a patient undergoing a targeted temperature management (TTM) therapy, in accordance with some embodiments.

FIG. 2 is a top view of a thermal pad of a TTM system, in accordance with some embodiments.

FIG. 3 is a block diagram of a system architecture adapted to support a thermal pad set recommendation system, in accordance with some embodiments.

FIG. 4 is a thermal pad set correlation table of the thermal pad set recommendation system of FIG. 3, in accordance with some embodiments.

FIG. 5 is a screen shot of a thermal pad set recommendation form of the thermal pad set recommendation system, in accordance with some embodiments.

FIG. 6 is flow chart for a process of determining the thermal pad set recommendation, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

The phrases “connected to” and “coupled with” refer to any form of interaction between two or more entities, including mechanical, electrical, magnetic, electromagnetic, fluid, signal, communicative (including wireless), and thermal interaction. Two components may be connected to or coupled with each other even though they are not in direct contact with each other. For example, two components may be coupled with each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

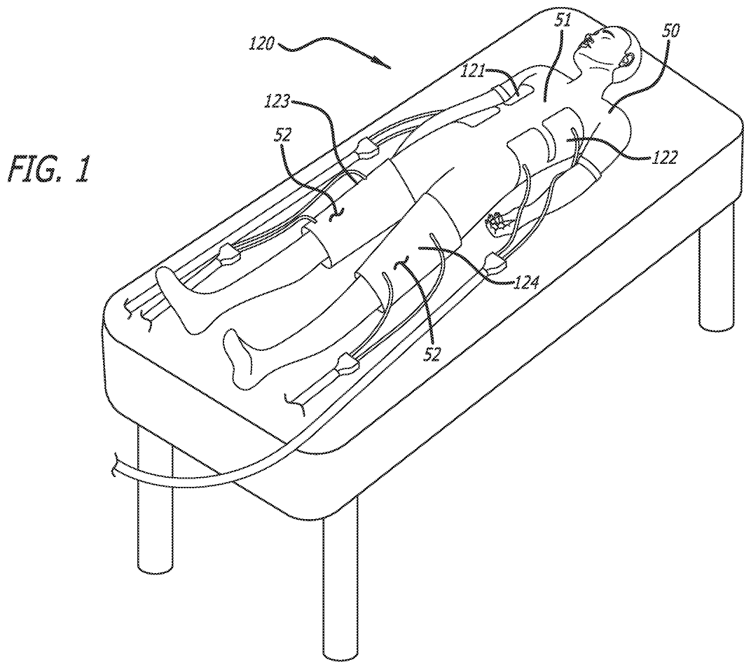

FIG. 1 illustrates a patient 50 undergoing a targeted temperature management (TTM) therapy, in accordance with some embodiments. In the illustrated embodiment, a thermal contact pad set 120 including four thermal contact pads 121, 122, 123, and 124 are applied to the patient 50. Torso pads 121 and 122 are applied to the torso 51 of the patient 50 such that each torso pad 121, 122 extends partially around the torso 51 of the patient 50. Thigh pads 123 and 124 are individually applied to each thigh 52 of the patient 50 such that each thigh pad 121, 122 extends at least partially around the thigh 52 of the patient 50. While the illustrated embodiment of the pad set 120 includes four pads, other embodiments may include one, two, three, four, five, six, or more thermal contact pads.

As shown, the pads are sized to cover a specified portion of the patient. For example, the torso pads may extend from the waist to the breast of the patient 50. Similarly, the thigh pads may extend from the groin area to the knee of the patient. As described below, different pad sets 120 may include pads of different sizes (i.e., dimensions) to accommodate different patient sizes.

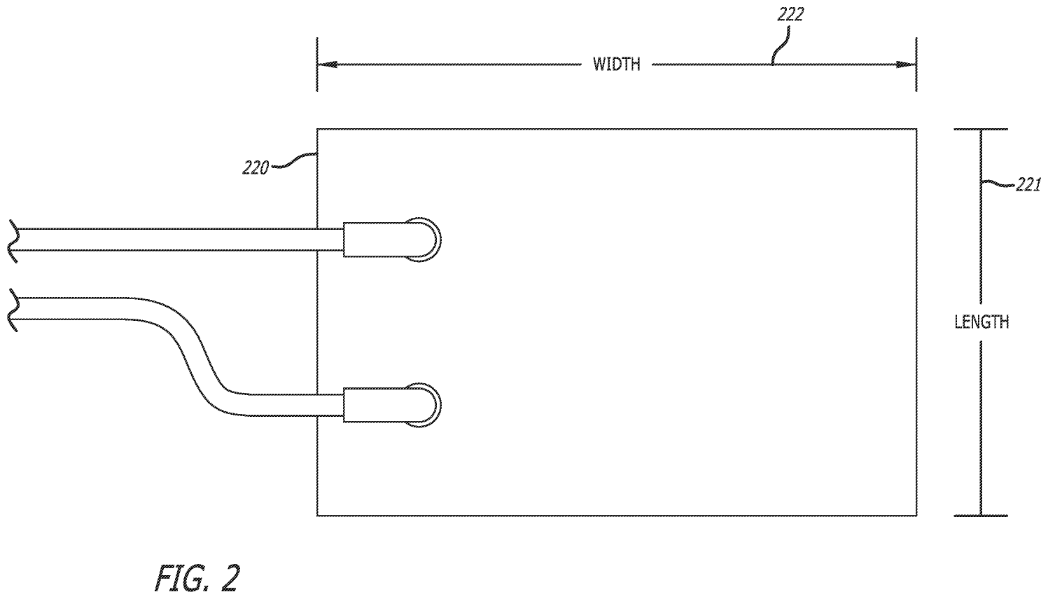

FIG. 2 is a top view of a thermal pad 220 which may be representative of any one pad of the pad set 120. In some embodiments, the thermal pad 220 may generally define a rectangular shape. As shown, the pad 220 defines a length dimension 221 which in use may be oriented parallel to a height of the patient 50. The pad 220 similarly defines a width 222 which may extend at least partially around a portion of the patient 50. In the illustrated embodiment, the length 221 and the width 222 may substantially define a fit for the pad 220 on the patient 50. For example, in the case of the thigh pads 123, 124, the length 221 may extend along a thigh length of the patient 50, i.e., between the groin area and the knee of the patient 50, and further in the case of the thigh pads 123, 124, the width 222 may extend partially or completely along a circumference of the thigh of the patient 50, i.e., around the thigh 52 of the patient 50. In some instances, the width 222 may exceed a circumference of the thigh 52 so that end portions of the width 222 may overlap each other. It should be understood that the rectangular shape of the pad 220 is not intended to be limiting and merely provides one illustrative embodiment. A pad as disclosed herein may take various shapes.

In the case of the torso pads 121, 122, the length 221 may extend along a length of the torso 51, i.e., from the breast of the patient 50 to a waste or hips of the patient 50. Similarly, the width 222 of the torso pad 121, 122 may extend partially around the torso 51 of the patient 50, i.e., extend along a portion of a torso circumference of the patient 50. As illustrated in FIG. 1, the torso pads 121, 122 may be positioned end to end such that the widths 222 of the torso pads 121, 122 extend around opposite portions of the torso 51. As such, when combined, the pads 121, 122 may extend substantially along the torso circumference of the patient 50. In some instances, the combined widths 222 of the torso pads 121, 122 may exceed the circumference of the torso 51 so that end portions of the torso pads 121, 122 may overlap each other.

In the illustrated embodiment, the length 221 and the width 222 may substantially define a fit for the pad 220 on the patient 50. As such, pads 220 of different lengths 221 and widths 222 may be provided to define the fit for patients 50 of different sizes. As patient sizes may range from neonates to extra-large adults, multiple pad sets 120 may be defined for use across the range of patient sizes. In use, the clinician may select a pad set 120 to fit a specific patient. While it may be possible for the clinician to obtain direct measurements of the patient 50 when selecting a pad size, obtaining direct measurements, e.g., thigh length or torso circumference, may be awkward or not logically feasible. In some instances, it may be necessary for the clinician to select a pad set 120 without having direct access to the patient 50.

In some instances, the clinician may select a pad set 120 in accordance with one or more available patient parameter values, e.g., a weight and/or height of the patient 50. However, as may be appreciated by one of ordinary skill, the patient dimensions that correlate with thermal pad dimensions may vary across patients having the same weight or height. For example, two patients having the same weight, may have different torso lengths or torso circumferences. As such, it may be advantageous for clinician to utilize a tool to more accurately select a pad set 120 in accordance with available patient parameter values.

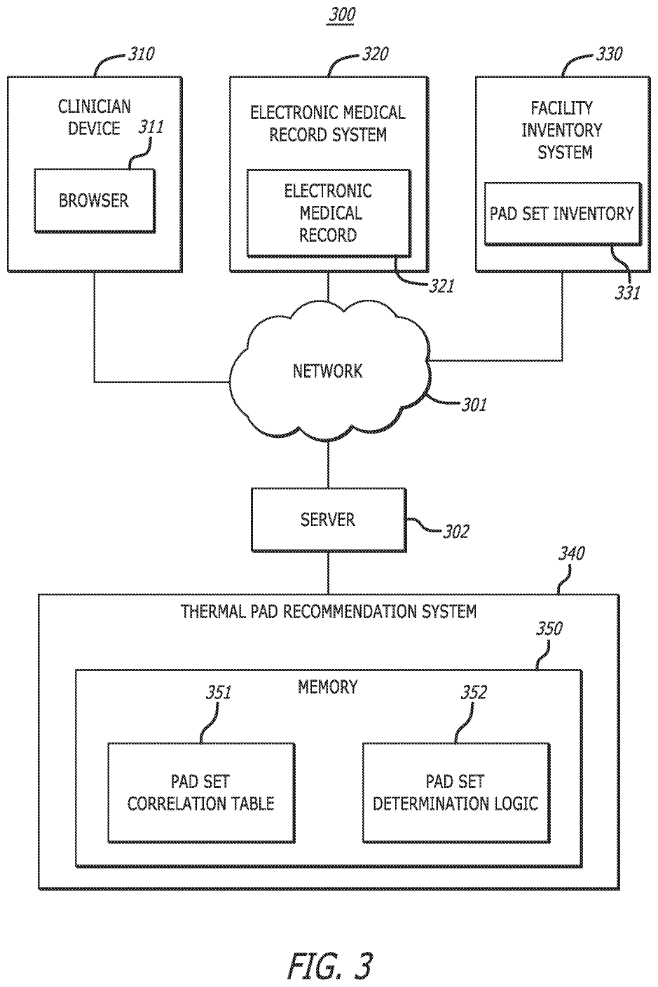

FIG. 3 illustrates a system architecture 300 adapted to support one embodiment of a thermal pad recommendation system (system) 340. The network 301 represents the communication pathways between the clinician device 310 and the system 340. In one embodiment, the network 301 is the Internet. The network can also utilize dedicated or private communication links (e.g., WAN, MAN, or LAN) that are not necessarily part of the Internet. The network uses standard communications technologies and/or protocols.

The server 302 may be a web server configured to present web pages or other web content, which form the basic interface to the clinician device 310. The clinician uses the clinician device 310 to access one or more web pages, and provide data to the pad recommendation system 340. In the context of this application, “data” is understood to include information about the patient 50, the pad set 120, a pad set inventory, and the like. For example, for information related to the patient 50, the data can include information such as weight, height, body fat percentage, pant waist size, pant inseam size, shoe size and the like. Also, for information about the pad set 120, the data can include the number of pads, types of pads, pad dimensions, part numbers, and the like.

The clinician device 310 is used by the clinician for interacting with the system 340. The clinician device 310 can be any device that is or incorporates a computer such as a personal computer (PC), a desktop computer, a laptop computer, a notebook, a smartphone, or the like. A computer is a device having one or more general or special purpose processors, memory, storage, and networking components (either wired or wireless). The device executes an operating system, for example, a Microsoft Windows-compatible operating system (OS), Apple OS X or iOS, a Linux distribution, or Google's Android OS. In some embodiments, the clinician device 310 may use a web browser 311, such as Microsoft Internet Explorer, Mozilla Firefox, Google Chrome, Apple Safari and/or Opera, as an interface to interact with the system 340. The clinician may provide patient parameter data to the system 340 by directly inputting values for defined patient parameters for the patient 50 via the clinician device 310.

The system architecture 300 may include access to an electronic medical record (EMR) system 320. The EMR system 320 may include an electronic medical record (EMR) 321 for the patient 50 and the EMR 321 may include one or more patient parameters. The patient parameters of the EMR 321 may include the patient's weight, the patient's height, and the patient's body-fat percentage. In some embodiments, the one or more patient parameters may be associated with an identifier or other key that may be provided on a patient wristband (e.g., a hospital wristband), a patient chart, etc. As one example, the identifier may be a barcode that is printed on the patient wristband or a patient chart such that scanning of the barcode provides at least a subset of the one or more patient parameters as input to the thermal pad recommendation system 340. The clinician device may include a barcode scanner or utilize a software application where execution thereof results in the scanning of the barcode. For instance, when the clinician device 310 includes a computer, the barcode scanner may be a peripheral device that couples to the laptop and may be considered an aspect of the clinician device 310. In other instances, such as when the clinician device 310 is a mobile device (e.g., phone or tablet), the clinician device 310 may include a software application (logic) that, upon execution, performs operations including scanning the barcode. In either instance, upon receiving the scanned barcode, the clinician device 310 may access the one or more patient parameters that are associated with the barcode and provide such to the thermal pad recommendation system 340.

The system architecture 300 may include access to a facility inventory system 330. The inventory system 330 may include a pad set inventory 331 defining a current availability within the facility of any one pad set 120 of a catalog of pad sets 120. In some instances, a pad selected from inventory may be relayed to the thermal pad recommendation system 340 and to the pad set determination logic 352, which as discussed below, may utilize machine learning techniques (or other artificial intelligence techniques) to determine a thermal pad size recommendation. Further, the size of the selected pad may be utilized in updating or refining the pad set determination logic 352 in order to improve accuracy of future recommendations. For example, the size of the selected pad may be utilized in re-training a machine learning model of the pad set determination logic 352.

In use, the clinician device 310 issues a request to the system 340 to obtain a recommendation for the pad set 120 to be used with a specified patient 50. In response, the system 340 provides a recommendation to the client 310 regarding the pad set 120 to be used with the specified patient 50 when performing the TTM therapy based on available patient parameter values. In some embodiments, the system 340 may also provide an alternative pad set recommendation.

Those of skill in the art will appreciate that the system architecture 300 may contain other modules that are not described herein. In addition, conventional elements, such as firewalls, authentication systems, payment processing systems, network management tools, load balancers, and so forth are not shown as they are not material to the invention. The system 340 may be implemented using a single computer, or a network of computers, including cloud-based computer implementations. The computers are preferably server class computers including one or more high-performance CPUs and 1G or more of main memory, and running an operating system such as LINUX or variants thereof. The operations of the system 111 as described herein can be controlled through either hardware or through computer programs installed in non-transitory computer storage and executed by the processors to perform the functions described herein. The system architecture 300 includes other hardware elements necessary for the operations described here, including network interfaces and protocols, input devices for data entry, and output devices for display, printing, or other presentations of data.

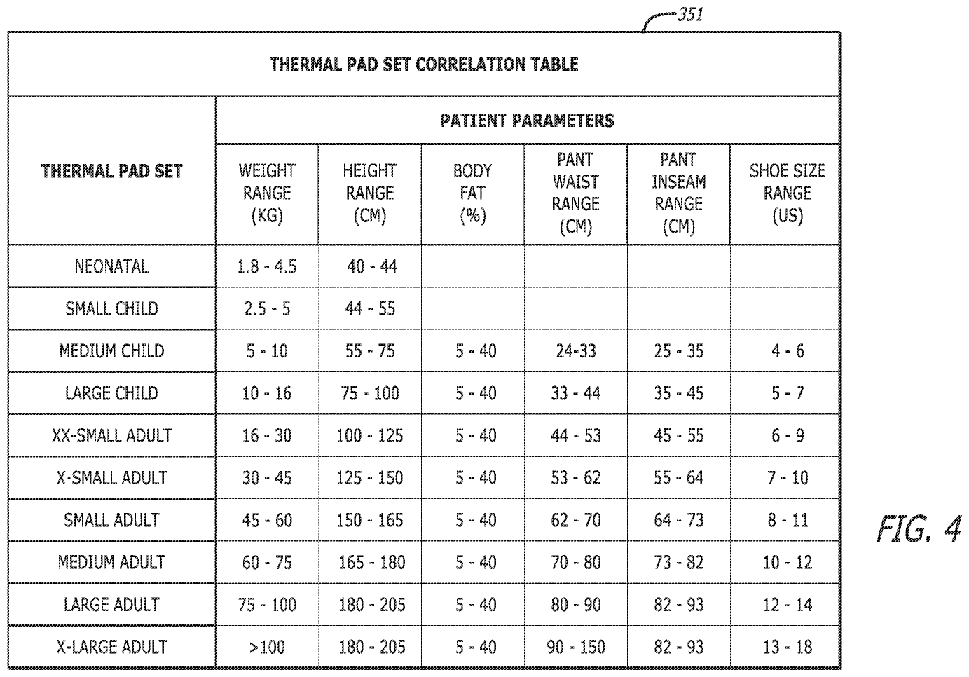

The system 340 includes a non-transitory computer readable storage medium 350 having a pad set correlation table 351 and a pad set determination logic 352 stored thereon, the logic 352 including a pad set determination algorithm. The pad set correlation table 351 associates value ranges of defined patient parameters with corresponding pad sets 120 as described in relation to FIG. 4. The pad determination logic 352 includes instructions such that when executed by one or more processors is configured to perform operations in accordance with providing the pad set recommendation to the clinician device 310 as further described below. In some embodiments, the non-transitory computer readable storage medium 350 may include multiple pad set correlation tables 351 for different genders. In other embodiments, data for all genders may be included in a single pad set correlation table 351.

In some embodiments, the system architecture 300 may include or have access to a three-dimensional (3D) body scanner (not shown) from which the system 340 may acquire one or more patient parameter values.

In some embodiments, a clinician may utilize a network device that includes a camera (e.g., a mobile phone or a tablet) and capture one or more images of the patient in lieu of an image captured by a 3D body scanner. In such embodiments, logic of the thermal pad recommendation system 340 may use computer-vision techniques to detect the patient and detect certain components of the patient's environment, such as a bed. In some embodiments, the environment components may include a device having specified length, such as a meter stick. Based on the detection of the patient and one or more environment components, the logic may determine dimensions of the patient such as an overall length of the patient body, the length of various portions of the patient body (e.g., length of torso, length arms, lengths of legs, etc.) and a width of various portions of the patient body.

In some embodiments, the pad set determination logic 352 may utilize machine learning techniques (or other artificial intelligence techniques) to determine a thermal pad size recommendation. For example, a machine learning model may be trained utilizing previously stored data indicating patient dimensions (e.g., manually entered height, weight, shoe size, body measurements, gender, etc., and/or captured images via a 3D body scanner or other cameras), corresponding selected thermal pad sizes and scores as to how the selected thermal pad size fit the patient. Thus, the trained machine learning model may be deployed by the thermal pad recommendation system to score various thermal pad sizes for data indicating patient dimensions, where a highest resultant score may indicate the recommendation.

FIG. 4 illustrates an exemplary pad set correlation table 351. The table 351 includes multiple pads sets 120 defined by sizes ranging from a neonatal size to an extra-large adult size. The table includes value ranges for defined patient parameters that correlate with each pad set 120. For example, as shown in the table 351, the “small adult” pad set 120 correlates typically to patients having a weight between 30 and 45 kg.

In some embodiments, the value ranges of the patient parameters represent typical value ranges for patients 50 across the patient parameters. For example, referring to the table 351, a patient having a weight between 30 and 45 kg may typically have a height between 155 and 165 cm, a body fat percentage between 5 and 40 percent, a pant waist size between 53 and 62 cm, a pant inseam between 64 and 73 cm, and a shoe size between 8 and 11 (US).

In some instances, actual patient parameter values may vary from the typical parameter value ranges in the table 351. For example, a patient weighing 46 kg may have height less than 150 cm. In such an instance, the patient's weight may correlate with the “small adult” pad set 120 and the patient's height may correlate with the “X-small Adult” pad set 120. As such, the system 340 may be of significant help to the clinician in resolving the disagreement and selecting a pad set 120.

Although not shown, the system 340 may include separate pad set correlation tables for male and female patients. In some instances, typical parameter value ranges for male patients may differ from typical parameter value ranges for female patients.

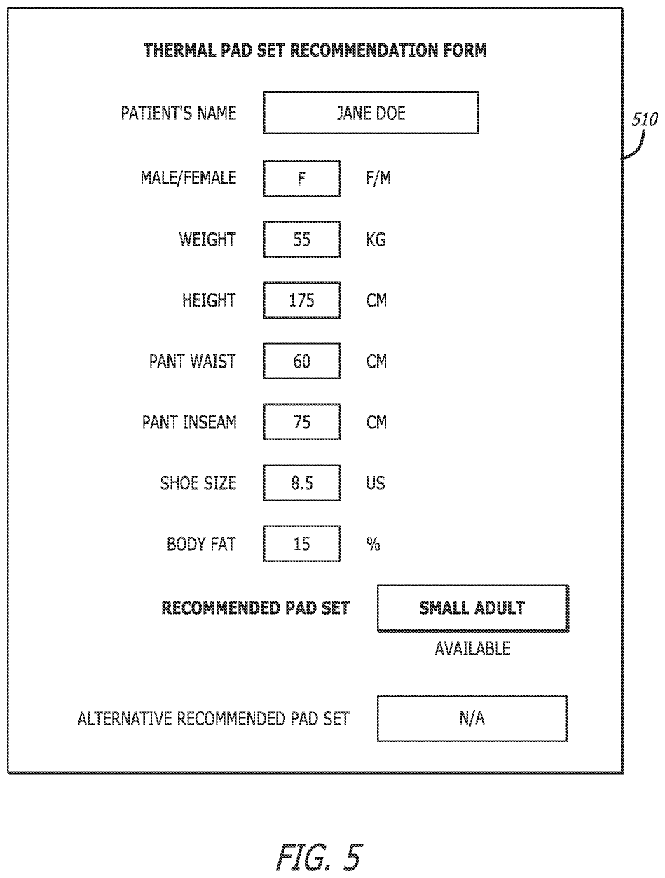

FIG. 5 illustrates a screen shot of an exemplary thermal pad set recommendation form (form) 510, in accordance with some embodiments. The form 510 includes patient parameters for which patient values may be acquired from the EMR 321. Such parameters may include the patient's gender, the patient's weight, the patient's height, and the patient's body fat percentage. The form 510 may also facilitate direct input of other patient parameter values by the clinician via the clinician device 310. These other parameters may include the patient's pant waist size, the patient's pant inseam size, and the patient's shoe size.

In some instances, the patient's pant waist size may more accurately correlate with the width 222 of the torso pads 121, 122 than the patient's weight. As such, in some instances when available, it may be advantageous to determine a pad set 120 in accordance with the patient's pant waist size. Similarly, the patient's pant inseam size may more accurately correlate with the length 221 of the thigh pads 123, 124 than the patient's height. As such, in some instances when available, it may be advantageous to determine a pad set 120 in accordance with the patient's pant inseam size.

The system 340 may display the recommended pad set 120 and an availability status on the clinician device 310. The system 340 may also display an alternative recommended pad set 120 if the recommended pad set 120 is unavailable in inventory. In some embodiments, the system 340 may display the recommended pad set, availability status, and an alternative recommended pad set 120 as a portion of the form 510.

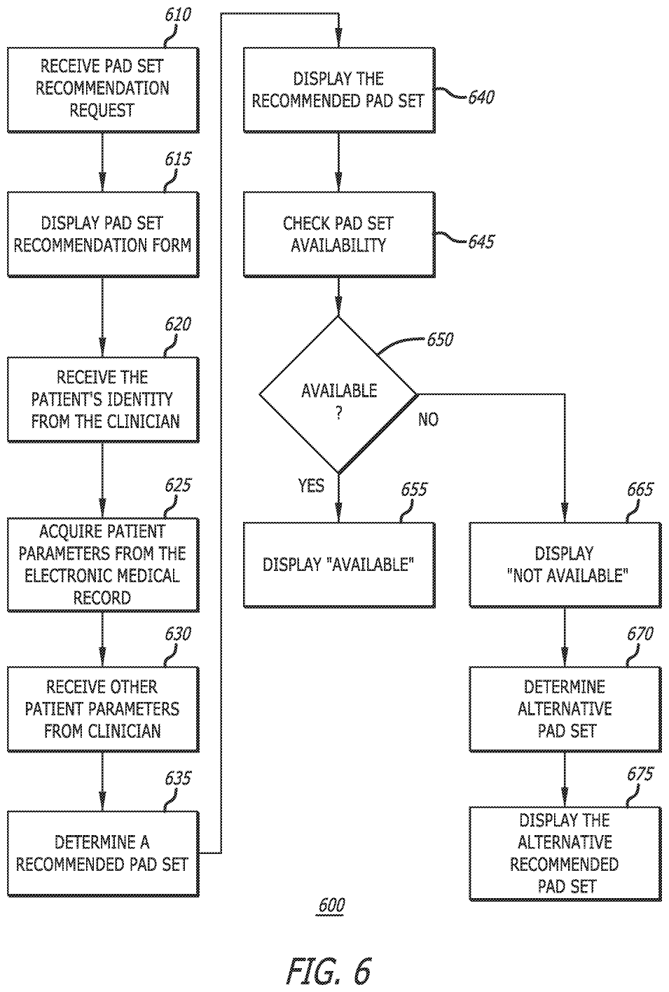

FIG. 6 illustrates a computer aided process 600 that may include the steps as described below. The logic 352 may receive a thermal pad recommendation request from the client 310 (step 610). In response, the logic 352 may display the form 510 so that the clinician 310 may input the patient's identity (step 615). The logic 352 may receive the patient's identity (e.g., the patient's name) as input by the clinician 310 (step 620). Having the patient's identity, the logic 352 may access the EMR system 320 and acquire any patient parameter values available on the patient's EMR 321 (step 625). The logic 352 may also receive any other patient parameter values as may be input by the clinician via the clinician device 310 (step 630). Having all available patient parameter values, the logic 352 may determine the recommended pad set 120 in accordance with the available patient parameter values (step 635) as further described below. Once the recommended pad set 120 is determined, the logic 352 may display the recommended pad set 120 on the clinician device 310 (step 640). The logic 352 may access the facility inventory system to determine if the recommended pad set 120 is available in inventory (step 645). If the recommended pad set 120 is available (step 650), the logic 352 may display a message accordingly (step 655).

If the recommended pad set 120 is not available (step 650), the logic 352 may display a message that the recommend pad set 120 is not available (step 665). The logic 352 may then determine an alternative pad set 120 from the pad sets 120 available in inventory (step 670) and display the alternative recommended pad set 120 on the clinician device 310 (step 675).

The determining step 635 may include operations as performed by the pad set determining logic 352. The logic 352 may determine the recommended pad set 120 from available patient parameter values on the form 510. In some instances, one or more patient parameter values may be omitted from the form 510, in which instances, the logic 352 may provide a recommended pad set 120 from the available patient parameter values on the form 510. In some embodiments, one patient parameter may provide a more accurate correlation to the pad set 120 than another patient parameter. For example, the patient's pant waist size may represent a patient's torso circumference more accurately than the patient's weight and thus may correlate more accurately to the pad set 120 than the patient's weight. In some instances, the patient's weight may correlate with a pad set 120 that is different (e.g., smaller or larger) than the pad set 120 that correlates with the patient's height. In some embodiments, the logic may apply a greater correlation significance to one patient parameter over another patient parameter. For example, in some embodiments, the logic 352 may apply a greater correlation significance to the patient's pant waist size and the pant inseam size, since the dimensions associated with these patient parameters may align more accurately with the dimensions of the pads, i.e., the length 221 and the width 222 (see FIG. 2).

In some instances, a patient parameter value may be close to an end of the parameter value range such that either of two pad sets 120 may equally correlate with the patient parameter value. In such an instance, the logic 352 may utilize a value of another patient parameter to determine which of the two pad sets 120 may define a better fit with the patient 50.

In some embodiments, the logic 352 may sequentially refine the pad set recommendation in accordance with ordered patient parameters. For example, the logic 352 may initially determine a recommended pad set 120 in accordance with a first patient parameter, (e.g., the patient's weight). Thereafter, the logic 352 may refine or alter the pad set recommendation in accordance with a second patient parameter, (e.g., the patient's height). Thereafter, the logic 352 may further refine or alter the pad set recommendation in accordance with a third patient parameter, (e.g., the patient's pant waste size). This pattern of refinement may continue until each of the available patient parameters have been used in determining the recommended pad set 120.

In some embodiments, the logic 352 may initially determine a recommended pad set 120 in accordance with a first set of patient parameters, (e.g., the patient parameters available from the EMR). Thereafter, the logic 352 may refine or alter the pad set recommendation in accordance with a second set of patient parameter, (e.g., the patient parameters input directly into the form 510 by the clinician).

A few examples of the pad set determining step 635 describe exemplary operations (e.g., algorithmic operations) of the logic 352, in accordance with some embodiments.

Example 1. The patient has a weight of 84 kg and a height of 185 cm. All other patient parameter values may be omitted on the form 510. In this example, the patient's weight is in the middle of the weight range for the “Large Adult” pad set 120, and the patient's height is in the middle of the height range for the “Large Adult” pad set 120. In response, the logic 352 may determine that the patient parameter values correlate with the “Large Adult” pad set 120.

Example 2. The patient has weight of 44 kg and a height of 160 cm. All other patient parameter values may be omitted on the form 510. In this example, the patient's weight is at the high end of the weight range for the “X-Small Adult” pad set 120, and the patient's height is at the high end of the height range for the “Small Adult” pad set 120. In response, the logic 352 may determine that patient parameter values correlate more accurately with the “Small Adult” pad set 120 over the “X-Small Adult” pad set 120 since using the “Small Adult” pad set 120 may provide sufficient length for the patient and the extra width may result in acceptable pad overlap.

Example 3. The patient has weight of 74 kg and a height of 150 cm. All other patient parameter values may be omitted on the form 510. In this example, the patient's weight is at the high end of the weight range for the “Medium Adult” pad set 120, and the patient's height is in the middle of the height range for the “Small Adult” pad set 120. In response, the logic 352 may determine that patient parameter values correlate more accurately with the “Medium Adult” pad set 120 over the “Small Adult” pad set 120 since using the “Medium Adult” pad set 120 may provide sufficient width to extend around the torso and thighs of the patient and the extra length of the thigh pads may acceptably extend into the knees of the patient and the extra length of torso pads may acceptably extend down to the hips of the patient.

Example 4. The patient has weight of 58 kg, a height of 155 cm, a pant waist size of 72 cm, and a pant inseam size of 70 cm. In this example, the patient's weight is at the high end of the weight range for the “Small Adult” pad set 120, the patient's height is in the middle of the height range for the “Small Adult” pad set 120, the patient's waist size is at the low end of the waist range for the “Medium Adult” pad set 120, and the patient's inseam is in the middle of the inseam range for the “Small Adult” pad set 120. In response, the logic 352 may determine that patient parameter values correlate more accurately with the “Medium Adult” pad set 120 over the “Small Adult” pad set 120 since the patient's waist size is a more accurate indication of the torso circumference than the patient's weight.

Example 5. The patient has weight of 74 kg, a height of 175 cm, a pant waist size of 75 cm, and a pant inseam size of 87 cm. In this example, the patient's weight is at the high end of the weight range for the “Medium Adult” pad set 120, the patient's height is at the high end of the height range for the “Medium Adult” pad set 120, the patient's waist size is in the middle of the waist range for the “Medium Adult” pad set 120, and the patient's inseam is in the middle of the inseam range for the “Large Adult” pad set 120. In response, the logic 352 may determine that patient parameter values correlate more accurately with the “Large Adult” pad set 120 since the patient's inseam is a more accurate indication of the thigh length than the patient's height.

Example 6. The patient has weight of 44 kg and a height of 160 cm. All other patient parameter values may be omitted on the form 510. In this example, the patient's weight is at the high end of the weight range for the “X-Small Adult” pad set 120, and the patient's height is at the high end of the height range for the “Small Adult” pad set 120. In response, the logic 352 may determine that patient parameter values correlate more accurately with the “Small Adult” pad set 120 over the “X-Small Adult” pad set 120 since using the “Small Adult” pad set 120 may provide sufficient length for the patient and the extra width may result in acceptable pad overlap. However, in this example, the logic 352 determines that the “Small Adult” pad set 120 is not available in inventory. As such, the logic 352 may define the “X-Small Adult” pad set 120 as the alternative pad set recommendation.

Example 7. Each of the height, weight and body fat percentage of the patient are unknown. However, a clinician is able to determine the size of the patient's waist via a pant waist size and the patient's shoe size. In the scenario in which the patient's pant waist size is 40 cm and has a shoe range of 6 (US children's size), the logic 352, upon receipt of such information, determines that a “Large Child” pad set 120 is appropriate. Here, the logic 352 determines the pant size corresponds to the “Large Child” pad set 120 and the shoe size corresponds to a “Medium Child” pad set 120. As a result, the logic 352 recommends the larger pad size.

Example 8. The patient has a weight of 100 kg and a height 150 cm. In this example, the patient's weight is at the high end of the weight range for the “Large Adult” pad set 120 and the patient's height is at the high end of the height range for the “X-Small Adult” pad set 120. However, in this situation, the logic 352 may recommend the “X-Large Adult” pad set 120. Such a recommendation may be based on experiential data that is included within the logic 352. In some embodiments, the logic 352 may include a trained machine learning model that provides a scoring of thermal pad set sizes based on input parameters disclosed above, where training is performed utilizing training data (e.g., scores of how a particular thermal pad set size fits on a patient, e.g., provided by clinicians).

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Embodiments of the invention may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible computer readable storage medium or any type of media suitable for storing electronic instructions, and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1-17. (canceled)

18. A system comprising:
one or more processors; and
a non-transitory computer-readable medium communicatively coupled to the one or more processors and having instructions stored thereon that, when executed by the one or more processors, cause performance of operations including:
receiving a request for a thermal pad set recommendation for an identified patient from a clinician device,
accessing an electronic medical record (EMR) for the patient,
retrieving one or more patient parameter values from the EMR,
determining a pad set recommendation according to the patient parameter values in combination with a pad set correlation table, and
displaying the pad set recommendation on the clinician device; and
a thermal contact pad set configured for application to a patient undergoing a targeted temperature management therapy and corresponding to the pad set recommendation including at least one of a torso pad or a thigh pad, wherein the torso pad.

19. The system of claim 18, wherein the pad set comprises at least one torso pad.

20. The system of claim 18, wherein the pad set comprises at least one thigh pad.

21. The system of claim 18, wherein the patient parameters comprise at least two of a gender of the patient, a weight of the patient, a height of the patient, or a body fat percentage of the patient.

22. The system of claim 18, wherein the patient parameters comprise at least two of a gender of the patient, a weight of the patient, a height of the patient, or a body fat percentage of the patient.

23. The system of claim 18, wherein the operations further comprise receiving one or more other patient parameters from the clinician device.

24. The system of claim 23, wherein the other patient parameters comprise at least one of the patient's pant waist size, pant inseam size, or shoe size.

25. The system of claim 23, wherein the other patient parameters comprise at least two of the patient's pant waist size, pant inseam size, or shoe size.

26. The system of claim 18, wherein determining the pad set recommendation comprises:
determining an initial pad set recommendation in accordance with a first set of patient parameters; and
determining a refined pad set recommendation in accordance with a second set of patient parameters in combination with the first set of patient parameters, and
wherein rendering the pad set recommendation on the clinician device comprises rendering refined pad set recommendation.

27. The system of claim 26, wherein the refined pad set recommendation is different from the initial pad set recommendation.

28. The system of claim 26, wherein the first set of patient parameters comprises one or more of the patient parameters, and wherein the second set of patient parameters comprises one or more of the other patient parameters.

29. The system of claim 26, wherein the first set comprises the patient's weight and/or the patient's height.

30. The system of claim 26, wherein the second set of patient parameters comprises the patient's pant waist size and/or the patient's pant inseam size.

31. The system of claim 18, wherein the operations further comprise:
accessing a facility inventory system; and
determining an availability of the recommended pad set in inventory.

32. The system of claim 31, wherein if the pad set is not available in inventory, the operations further comprise:
determining an alternative pad set; and
displaying the alternative pad set on the clinician device.

33. The system of claim 18, wherein determining the recommended thermal pad set according to the patient parameter values is performed using a trained machine learning model, wherein the trained machine learning model receives as input the one or more patient parameter values and provides one or more resultant scores.

34. The system of claim 33, wherein a highest resultant score is provided as the recommended thermal pad set.

35-51. (canceled)

52. The system of claim 18, wherein the instructions that, when executed by the one or more processors, cause performance of further operations including:
initiating the targeted temperature management therapy including causing a temperature of a fluid to be adjusted to a target temperature and the fluid to be delivered to the thermal contact pad set resulting in an adjustment of a patient temperature toward a target patient temperature.
